(19) United States
(12) Patent Application Publication
(10) Pub. No.: US 2020/0337907 A1
(43) Pub. Date: Oct. 29, 2020

(54) NEGATIVE PRESSURE WOUND THERAPY ARTICLE

(71) Applicant: 3M INNOVATIVE PROPERTIES COMPANY, St. Paul, MN (US)

(72) Inventors: Robert T. Fitzsimons, Jr., Minneapolis, MN (US); Bryan A. Baker, Minneapolis, MN (US); Jana Ninkovic, St. Paul, MN (US); Jie Liu, Woodbury, MN (US); Federica Sgolastra, St. Paul, MN (US); Minghua Dai, Plymouth, MN (US); Wei Zhang, Woodbury, MN (US); Amy K. McNulty, Stillwater, MN (US)

(21) Appl. No.: 16/759,090

(22) PCT Filed: Oct. 30, 2018
(86) PCT No.: PCT/IB2018/058493

§ 371 Date: Apr. 24, 2020

Related U.S. Application Data
(60) Provisional application No. 62/598,697, filed on Dec. 14, 2017.
(60) Provisional application No. 62/579,476, filed on Oct. 31, 2017.

(51) Int. Cl. A61F 13/02; A61F 13/00; A61L 15/22

(52) U.S. Cl. CPC A61F 13/0216; A61F 13/00068; A61F 2013/00174; A61F 2013/00336; A61L 15/22

(57) ABSTRACT

An article including: a network of interconnected polymeric strands; wherein each of the interconnected polymeric strands has a first surface adapted to contact a tissue site; wherein none of the interconnected polymeric strand has a feature extending from the first surface of the interconnected polymeric strands; wherein at least one of the interconnected polymeric strands is non-linear; and a plurality of openings between adjacent interconnected polymeric strands; wherein the article is a negative pressure wound therapy article

### BACKGROUND

Clinical studies and practice have shown that providing a negative pressure in proximity to a tissue site promotes the growth of new tissues at the tissue site. The application of negative pressure is successful in treating wounds. This treatment (frequently referred to in the medical community as “negative pressure wound therapy (NPWT),” “reduced pressure therapy,” or “vacuum therapy”) provides a timber of benefits, including faster healing and increased formulation of granulation tissue. Typically, reduced pressure is applied to tissues through a foam, a pad or other manifolding device, such as gauze. The manifolding device typically contains cells, pores or other openings that are capable of distributing reduced pressure to the tissue and channeling fluids that are drawn from the tissue. The porous pad often is incorporated into a dressing having other components that facilitate treatment.

Other know NPWT articles are discussed in U.S. Pat. Nos. 7,494,482; 8,057,447; 8,889,243 and 9,107,989.

### SUMMARY

In one aspect, the present disclosure provides an article comprising: a network of interconnected polymeric strands; wherein each of the interconnected polymeric strands has a first surface adapted to contact a tissue site; wherein none of the interconnected polymeric strand has a feature extending from the first surface of the interconnected polymeric strands; wherein at least one of the interconnected polymeric strands is non-linear; and a plurality of openings between adjacent interconnected polymeric strands; wherein the article is a negative pressure wound therapy article.

In another aspect, the present disclosure provides a system including the article of present disclosure and a reduced pressure source fluidly connected to the opening of the article to deliver the reduced pressure through the opening, and to the tissue site.

In another aspect, the present disclosure provides a method, including providing the article of present disclosure and positioning the article on a wound.

Various aspects and advantages of exemplary embodiments of the present disclosure have been summarized. The above Summary is not intended to describe each illustrated embodiment or every implementation of the present disclosure. Further features and advantages are disclosed in the embodiments that follow. The Drawings and the Detailed Description that follow more particularly exemplify certain embodiments using the principles disclosed herein.

### BRIEF DESCRIPTION OF THE DRAWINGS

The disclosure may be more completely understood in consideration of the following detailed description of various embodiments of the disclosure in connection with the accompanying figures, in which:

FIG. 1 illustrates an article according to an embodiment of the present invention.

FIG. 2 illustrates a top view of an article according to an embodiment of the present invention,

FIG. 3 illustrates a schematic cross-section side view of an article according to an embodiment of the present invention.

FIG. 4 illustrates a reduced pressure treatment system according to an embodiment of the present invention.

While the above-identified drawings, which may not be drawn to scale, set forth various embodiments of the present disclosure, other embodiments are also contemplated, as noted in the Detailed Description. In all cases, this disclosure describes the presently disclosed invention by way of representation of exemplary embodiments and not by express limitations. It should be understood that numerous other modifications and embodiments can be devised by those skilled in the art, which fall within the scope and spirit of this disclosure.

### DETAILED DESCRIPTION

Before any embodiments of the present disclosure are explained in detail, it is understood that the invention is not limited in its application to the details of use, construction, and the arrangement of components set forth in the following description. The invention is capable of other embodiments and of being practiced or of being carried out in various ways that will become apparent to a person of ordinary skill in the art upon reading the present disclosure. Also, it is understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It is understood that other embodiments may be utilized and structural or logical changes may be made without departing from the scope of the present disclosure.

As used in this Specification, the recitation of numerical ranges by endpoints includes all numbers subsumed within that range (e.g. 1 to 5 includes 1, 1.5, 2, 2.75, 3, 3.8, 4, and 5, and the like).

Unless otherwise indicated, all numbers expressing quantities or ingredients, measurement of properties and so forth used in the Specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

The article of the present application is well suited to promote tissue growth at the tissue site vet prevent in-growth of new tissue into the article. The article of the present application can help to deliver a significant portion of microstrain to the wound site by the architecture of the article, for example, the surface morphology of the article and thus may allow for lower pressure settings for NPWT to be used (for example, −75 mmHg vs −125 mmHg). This may allow a longer battery life of the NPWT system and the use of smaller pumps for the NPWT.

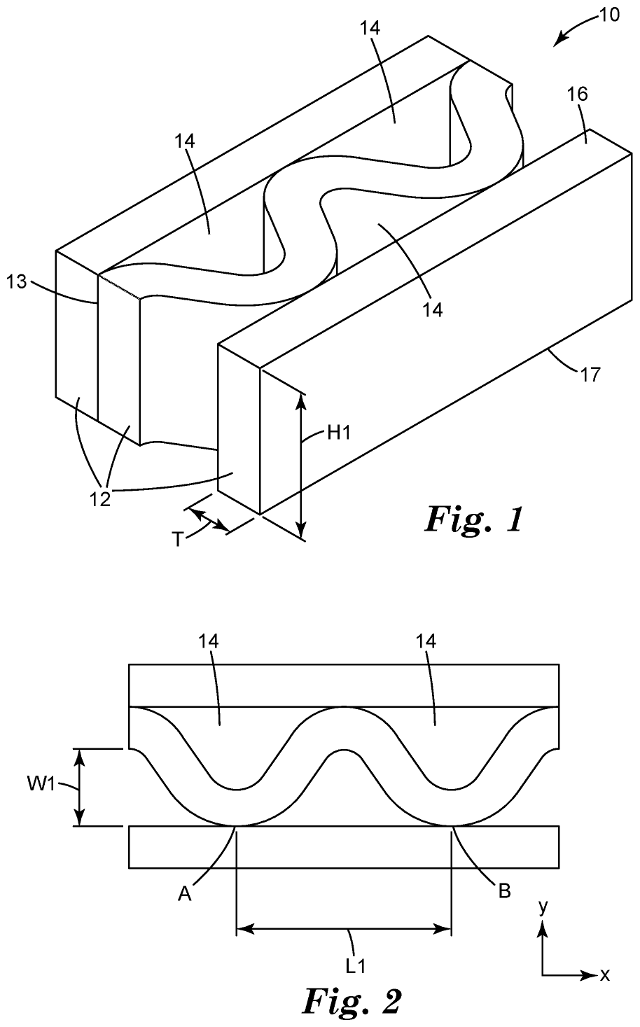

Referring to FIG. 1, a NPWT article according to an embodiment of the present invention includes a network of interconnected polymeric strands, or sheets 12 and a plurality of openings 14 between adjacent polymeric strands. Polymeric strands 12 can be connected at connections 13. Typically, there are a plurality of connections 13 between adjacent strands. Polymeric strands 12 have a tissue contact surface 16 as a first surface and a second surface 17 opposite the first surface. The first surface 16 does not have a feature, or a protrusion that extend from the first surface 16. In other words, none of the interconnected polymeric strand has a feature extending from the first surface of the interconnected polymeric strands. The openings 14 form or provide open fluid channels from the first surface 16 of the network of polymeric strands to a second surface opposite first surface 16. Through the open fluid channels, openings 14 are typically used to allow reduced pressure applied to a tissue site.

Referring more specifically to FIG. 1, the height, H1, of each polymeric strand 12 may be up to 2000 micrometers, up to 1500 micrometers, up to 1000 micrometers, up to 500 micrometers, or up to 400 micrometers. In some embodiments, the height, H1, of each polymeric strand 12 may be no less than 100 micrometers, no less than 200 micrometers, or no less than 300 micrometers. In some embodiments, the height, H1, of each polymeric strand 12 may be between 100 and 2000 micrometers, between 200 and 1500 micrometers, between 300 and 1000 micrometers, between 300 and 500 micrometers or between 300 and 400 micrometers. In some embodiments, the thickness, T, of each polymeric strand 12 may have an average width up to 500 micrometers, up to 400 micrometers, or up to 250 micrometers. In some embodiments, the thickness, T, of each polymeric strand 12 may have an average width no less than 10 micrometers. In some embodiments, the thickness, T, of each polymeric strand 12 may have an average width in a range from 10 micrometers to 500 micrometers, from 10 micrometers to 400 micrometers, or 10 micrometers to 250 micrometers. In some embodiments, the article comprising interconnected polymeric strands has an average thickness not greater than 5 mm. In one embodiment of the present invention, the height and thickness of the interconnected polymeric strands 12 is uniform for a particular article 10. In other embodiments, the height and thickness of the interconnected polymeric strands 12 may be different. For example, the interconnected polymeric strands 12 having different height. Similarly, thickness of the interconnected polymeric strands 12 may vary. In some, embodiments, the interconnected polymeric strands 12 may have a range of thicknesses, for example, the interconnected polymeric strands 12 tends to be thinnest where it abuts an opening.

In some embodiments, the article comprising interconnected polymeric strands has a thickness up to 2 mm, up to 1 mm, up to 500 micrometers, up to 250 micrometers, up to 100 micrometers, up to 75 micrometers, up to 50 micrometers, or up to 25 micrometers. In some embodiments, the article comprising interconnected polymeric strands has a thickness no less than 10 micrometers. In some embodiments, the article comprising interconnected polymeric strands has a thickness in a range from 10 micrometers to 2 mm, 10 micrometers to 1 mm, 10 micrometers to 750 micrometers, 10 micrometers to 500 micrometers, 10 micrometers to 250 micrometers, 10 micrometers to 100 micrometers, 10 micrometers to 75 micrometers, 10 micrometers to 50 micrometers, or 10 micrometers to 25 micrometers. In some embodiments, the article comprising interconnected polymeric strands has an average thickness in a range from 250 micrometers to 5 mm.

In some embodiments, at least one of the interconnected polymeric strands 12 may be non-linear. In some embodiments, at least 25% of the interconnected polymeric strands 12 may be non-linear. In some embodiments, at least 50% of the interconnected polymeric strands 12 may be non-linear. In some embodiments, at least 75% of the interconnected polymeric strands 12 may be non-linear. In some embodiments, essentially all (more than 90%, but less than 100%) the interconnected polymeric strands 12 may be non-linear. in some embodiments, all of the interconnected polymeric strands 12 may be non-linear. In some embodiments, the non-linear polymeric strand may have a shape of a curve. In some embodiments, the non-linear polymeric strand may have a shape of a sinusoidal curve. In some embodiments, the non-linear polymeric strand may have a shape of a sinusoidal curve. in other embodiments, at least one of the interconnected polymeric strands 12 may be linear. In some other embodiments, 25% to 75% of the interconnected polymeric strands 12 may be linear. In some other embodiments, 50% to 75% of the interconnected polymeric strands 12 may be linear. In certain embodiments, the network of interconnected polymeric strands may include alternating non-linear polymeric strands and linear polymeric strands, as shown in FIG. 2. in some embodiments, the interconnected polymeric strands 12 are oriented in the same direction, for example, x direction as illustrated in FIG. 2. In some embodiments, the interconnected polymeric strands 12 do not substantially cross over each other (i.e., at least 50 (at least 55, 60, 65, 70, 75, 80, 85, 90. 95, 99, or even 100) percent by number do not cross over each other).

In some embodiments, aspect ratio (a ratio of the length to the width) of the openings 14 may be greater than 1:1, 1.5:1, 2:1, 3:1 or 5:1. In some embodiments, aspect ratio (a ratio of the length to the width) of the openings 14 may be in a range from 1:1 to 100:1, 1:1 to 75:1, 1:1 to 50:1, 1:1 to 25:1, 2:1 to 100:1 2:1 to 75:1, 2:1 to 50:1. 2:1 to 25:1, or 2:1 to 10:1. The length, L1 of an opening 14 illustrated in FIG. 2 is the longest lateral distance parallel to x direction, for example, the length between connections A and C. If the non-linear polymeric strand has a shape of a sinusoidal curve, the length of the opening 14 equals the wavelength of the sinusoidal curve. The width, W1, of an opening 14 illustrated in FIG. 2 is the longest distance parallel to y direction. If the non-linear polymeric strand has a shape of a sinusoidal curve, the width of the opening 14 equals two times amplification of the sinusoidal curve. Openings 14 of the article may have a range of L1 and W1 values as a result in part of variable spacing of the connections A and B. In some embodiments, the openings have widths, W1, up to 10 mm, up to 1 mm or up to 0.5 mm. In some embodiments, the openings have widths, W1, at least 5 micrometers or at least 10 micrometers. In some embodiments, the openings have widths, W1, in a range from 5 micrometers to 1 mm or from 10 micrometers to 0.5 mm. In some embodiments, the openings have lengths, L1, up to 10 mm or up to 1 mm. In some embodiments, the openings have lengths, L1, at least 100 micrometers. In some embodiments, the openings have lengths, L1, in a range from 100 micrometers to 10 mm or from 100 micrometers to 1 mm. FIGS. 1 and 2 are idealized illustrations of one embodiment of the present application. In some embodiments, the openings 14 may have irregularly formed perimeters. This can mean that the openings have irregular shapes (that is, no lines of symmetry). They may have edges that are not smooth (e.g., jagged or feathery edges). Irregularly formed openings can also have a variety of thicknesses of the polymeric strands surrounding the openings.

In some embodiments, openings 14 may have any suitable shape, for example, a shape selected from shapes of ellipse, oval, pointed oval (or lens), diamond, ½ ellipse, ½ oval, ½ lens, triangle, etc. In some embodiments, the openings of the mechanical fastening nets described herein have at least two pointed ends. In some embodiments, at least some of the openings are elongated with two pointed ends. In some embodiments, at least some of the openings are elongated with two opposed pointed ends. In some embodiments, at least some of the openings are ovals.

In some embodiments, the article described herein have a total open area for each of the first and second, generally opposed surfaces of not greater than 50 (in some embodiments, not greater than 45, 40, 35, 30, 25, 20, 15, 10, 5, 4 3, 2, 1, 0.75, 0.5, 0.25, or even not greater than 0.1) percent of the total area of the respective surface. In some embodiments, for at least a majority of the openings of the article described herein, the maximum area of each opening is not greater than is 5 (in some embodiments, not greater than 2.5, 2, 1, 0.5, 0.1, 0.05, 0.01. 0.075, or even not greater than 0.005)mm². Individual openings range from 0.005 mm²to 5 mm². In some embodiments, the article according to the present disclosure have in a range from 50,000 to 6,000,000 (in some embodiments, 100,000 to 6,000,000, 500,000 to, 6,000,000, or even 1,000,000 to 6,000.000) openings/m².

In some embodiments, the tensile strength of the article parallel to x direction is greater than the tensile strength of the article parallel to y direction. Therefore, the article is easier to be stretched in y direction than in x direction. In some embodiments, the tensile strength of the article parallel to x direction is at least 2.23 MPa, at least 2.25 MPa, at least 2.5 MPa or at least 3.0 MPa. In some embodiments, the tensile strength of the article parallel to x direction is up to 5.42 MPa, up to 5.3 MPa, up to 5.0 MPa, or up to 4.5 MPa. In some embodiments, the tensile strength of the article parallel to x direction is from 2.23 MPa to 5.42 MPa, from 2.5 MPa to 5.0 MPa or from 3.0 MPa to 4.5 MPa. The Young's modulus of the article is up to 10.6 MPa, up to 10.0 MPa, up to 9.0 MPa, or up to 8.0 MPa. The Young's modulus of the article is at least 3.85 MPa, at least 4.0 MPa has a range from 3.85 MPa to 10.6 MPa in a direction parallel to x direction.

The presence and sizing of the opening allow opening to distribute reduced pressure to the tissue site. In addition to distributing reduced pressure to the tissue site, the article 10 also serves to impart stresses and strains to the tissue site similar to those seen with cellular foam that traditionally has been used in reduced pressure systems. Other materials sometimes used in reduced pressure systems, such as gauze, do not have this effect on tissue. Unbound by the theory, the stresses and strains created by the article 10 are believed to cause micro-deformation of existing tissues and plays a significant role in the generation of new tissues at the tissue site. The amount of stress and strain imparted to a tissue site is determined by the amount of reduced pressure supplied to the tissue site and the surface morphology of the article that contacts the tissue site. As reduced pressure is applied, portions of the tissue site are pulled against the article 10, which results in the development of stresses and strains within the tissue. In some embodiments, the article of the present disclosure can be a mechanical fastening net or a mechanical fastening sheet.

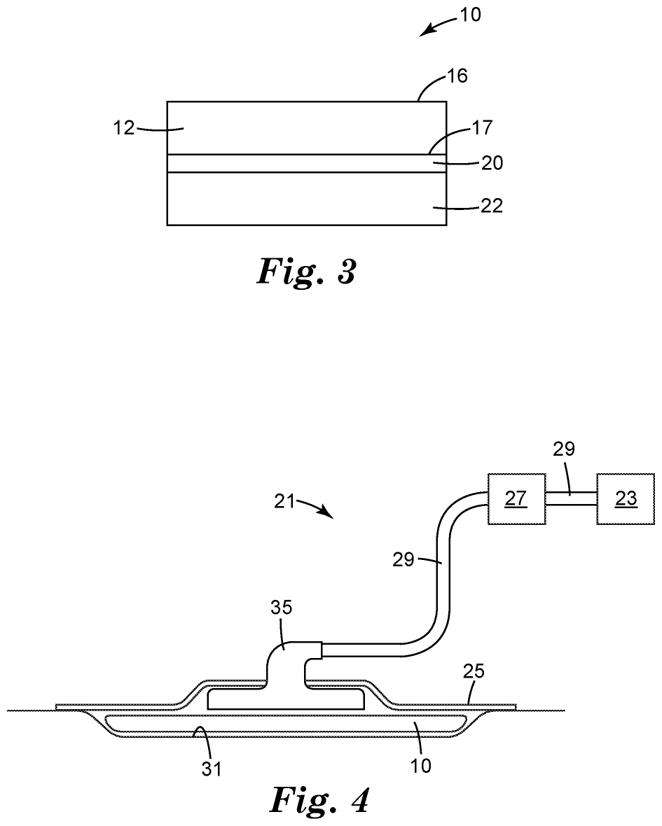

Referring to FIG. 3, in some embodiments, the article 10 may further include an adhesive layer 20 in contact with the second surface 17 of the interconnected polymeric strands 12. Suitable adhesive for use in the adhesive layer 20 of the article 10 can include any adhesive that provides acceptable adhesion to skin and is acceptable for use on skin (e.g., the adhesive should preferably be non-irritating and non-sensitizing). Suitable adhesives can be pressure sensitive and in certain embodiments have a relatively high moisture vapor transmission rate to allow for moisture evaporation. Suitable pressure sensitive adhesives include those based on acrylates, urethane, hyrdogels, hydrocolloids, block copolymers, silicones, rubber based adhesives (including natural rubber, polyisoprene, polyisobutylene, butyl rubber etc.) as well as combinations of these adhesives. The adhesive component may contain tackifiers, plasticizers, rheology modifiers as well as active components including for example an antimicrobial agent. Suitable adhesive can include those described in U.S. Pat. Nos. 3,389,827; 4,112,213; 4,310,509; 4,323,557; 4,595,001; 4,737,410; 6,994,904 and International Publication Nos. WO 2010/056541; WO 2010/056543 and WO 2014/149718, the disclosures of which are hereby incorporated by reference.

The article 10 may further include a cellular foam or another material 22 in contact with the adhesive layer, the adhesive layer in between the network of interconnected polymeric strands and the cellular foam or another material 22. In some other embodiments, the cellular foam or another material 22 that is positioned adjacent to or attached to the surface 17 of the interconnected polymeric strands opposite the features 18. The use of a cellular foam or other material 22 increases the ability of the reduced pressure conduit 29 or the distribution adapter 35 to deliver and distribute reduced pressure to the article 10. The features 18 and interconnected polymeric strands serve as a barrier to new tissue growth entering pores of the cellular foam or other material.

Referring to FIG. 4, a reduced pressure treatment system 21 according to an embodiment of the present invention includes a reduced pressure dressing, or article 10 fluidly connected to a reduced pressure conduit 29. The reduced pressure conduit 29 is fluidly connected to a reduced pressure source 23 such as a vacuum pump or another source of suction. The article 10 is placed against a tissue site 31 of a patient and is used to distribute a reduced pressure provided by the reduced pressure source 23. Typically, reduced pressure is maintained at the tissue site by placing an impermeable or semi-permeable cover 25 over the article 10 and the tissue site 31. The reduced pressure also serves to draw wound exudates and other fluids from the tissue site 31. A canister 27 may be fluidly connected to the reduced pressure conduit 29 and disposed between the article 10 and the reduced pressure source 23 to collect the fluids drawn from the tissue site 31. A distribution adapter 35 may be connected to the reduced pressure conduit 29 and positioned on the article 10 to aid in distributing the reduced pressure to the article 10.

In some embodiments, the interconnected polymeric strands 12 can include an elastomeric polymer. Elastomeric polymer can be any suitable elastomeric polymer, including but not limited to polyolefins and polyurethanes. In some embodiments, elastomeric polymer can be a medical grade material that is relatively impermeable to fluid flow. Alternatively, elastomeric polymer can be a semi-permeable material that allows select fluids or amounts of fluids to pass. In some embodiments, the composition of interconnected polymeric strands 12 may be formed from different materials.

Sonic embodiments of the present wound-treatment methods can include positioning the article of present disclosure on a wound of a patient and applying a reduced pressure to the wound through the article (e.g., through the openings). Some embodiments further comprise: coupling a drape to skin adjacent the wound such that the drape covers the article and the wound, and forms a space between the drape and the wound. In some embodiments, positioning the article on the wound can include placing the article over the wound on the first surface facing the wound. In some embodiments, applying the reduced pressure to the wound comprises activating a vacuum source (e.g., reduced pressure source 23 of FIG. 4) that is coupled to the article. Some embodiments comprise: delivering a fluid to the wound through the article. In some embodiments, delivering a fluid comprises activating a fluid source that is coupled to the article.

The following embodiments are intended to be illustrative of the present disclosure and not limiting.

### Embodiments

Embodiment 1 is an article, comprising: a network of interconnected polymeric strands; wherein each of the interconnected polymeric strands has a first surface adapted to contact a tissue site; wherein none of the interconnected polymeric strand has a feature extending from the first surface of the interconnected polymeric strands; wherein at least one of the interconnected polymeric strands is non-linear; and a plurality of openings between adjacent interconnected polymeric strands; wherein the article is a negative pressure wound therapy article.

Embodiment 2 is the article of embodiment 1, wherein at least one of the interconnected polymeric strands is linear.

Embodiment 3 is the article of any one of embodiments 1 to 2, wherein the network comprises alternating non-linear polymeric strands and linear polymeric strands.

Embodiment 4 is the article of any one of embodiments 1 to 3, wherein the non-linear polymeric strand has a sinusoidal curve.

Embodiment 5 is the article of any one of embodiments 1 to 4, wherein the tensile strength of the article parallel to x direction is greater than the tensile strength of the article parallel to y direction.

Embodiment 6 is the article of any one of embodiments 1 to 5, wherein the tensile strength of the article parallel to x direction is more than 2.23 Mpa.

Embodiment 7 is the article of any one of embodiments 1 to 6, wherein the polymeric strands comprises an elastomeric polymer.

Embodiment 8 is the article of embodiment 7, wherein the elastomeric polymer is selected from polyolefins or polyurethanes.

Embodiment 9 is the article of any one of embodiments 1 to 8, wherein aspect ratio of the openings is greater than 1:1.

Embodiment 10 is the article of any one of embodiments 1 to 9, wherein essentially all the interconnected polymeric strands are non-linear.

Embodiment 11 is a system, comprising: the article of any one of embodiments 1 to 10; and a reduced pressure source fluidly connected to the opening of the article to deliver the reduced pressure through the opening, and to the tissue site.

Embodiment 12 is a method, comprising: providing the article of any one of embodiments 1 to 10; and positioning the article on a wound.

Embodiment 13 is the method of embodiment 12, wherein positioning the article on the wound comprises continuously positioning the article on the wound for more than 3 days.

Embodiment 14 is the method of embodiment 12, wherein positioning the article on the wound comprises continuously positioning the article on the wound for more than 4 days.

Embodiment 15 is the method of embodiment 12, wherein positioning the article on the wound comprises continuously positioning the article on the wound for more than 5 days.

Embodiment 16 is the method of embodiment 12, wherein positioning the article on the wound comprises continuously positioning the article on the wound for more than 6 days.

Embodiment 17 is the method of embodiment 12, further comprising coupling a reduced pressure source to the article.

Embodiment 18 is the method of embodiment 12, further comprising applying a reduced pressure to the wound through the article.

Embodiment 19 is the method of embodiment 18, wherein applying the reduced pressure to the wound comprises activating the reduced pressure source coupled to the article.

Embodiment 20 is the method of any one of embodiments 16 to 19, wherein positioning the article on the wound comprises placing the article over the wound with the first surface facing the wound.

Embodiment 21 is a method for treating a wound comprising placing an article of any one of the Embodiments 1-10 on the surface of the wound, covering at least a portion of the article with a foam pad, and applying a negative pressure to the wound.

Embodiment 22 is the method of Embodiment 21, wherein one surface of the article is in contact with the wound and the opposite surface of the article is in contact with the foam pad.

Embodiment 23 is the method of Embodiments 21-22, wherein the foam is an open-cell foam.

Embodiment 24 is the method of Embodiments 21-23, wherein the foam is a polyurethane foam.

Embodiment 25 is the method of Embodiments 21-24, wherein the surface of the foam pad opposite from the article is covered by an impermeable or semi-permeable film.

Embodiment 26 is the method of Embodiment 25, wherein the impermeable or semi-permeable film is adhesively attached to the skin surface adjacent the wound.

Embodiment 27 is the method of Embodiments 25-26, wherein the film forms an occlusive dressing.

Embodiment 28 is the method of Embodiments 21-27, wherein the negative pressure is supplied by a vacuum source.

Embodiment 29 is the method of Embodiment 28, wherein the vacuum source is a vacuum pump.

Embodiment 30 is the method of Embodiments 28-29, wherein the foam pad is fluidly connected to the vacuum source.

Embodiment 31 is the method of Embodiments 21-30, wherein the article is a net.

Embodiment 32 is the method of Embodiment 31, wherein the net is in the form of a sheet.

Embodiment 33 is the method of Embodiment 32, wherein one or more sheets of netting are optionally placed between the article (i.e. first sheet of netting) and the foam pad.

Embodiment 34 is the method of Embodiments 21-33, wherein the article serves as a barrier to new tissue growth entering pores of the foam pad.

Embodiment 35 is a kit for treating a wound comprising the article of any one of the embodiments 1-10 and a foam pad.

Embodiment 36 is the kit of Embodiment 35, wherein the foam pad is an open-cell foam pad.

Embodiment 37 is the article of embodiment 1 to 10, wherein each of the interconnected polymeric strands has a second surface opposite the first surface and the article comprises an adhesive layer in contact with the second surface of the interconnected polymeric strands.

Embodiment 38 is the article of Embodiment 37, further comprising a cellular foam material in contact with the adhesive layer, the adhesive layer in between the network of interconnected polymeric strands and the cellular foam material.

The following working examples are intended to be illustrative of the present disclosure and not limiting.

### EXAMPLES

All animal studies were conducted with approval of the internal Institutional Animal Care and Use Committee (IACUC) in compliance with the United States Guide for the Care and Use of Laboratory Animals and the United States Animal Welfare Act (9CFR).

### EXAMPLE 1

A polyolefin net was prepared using ENGAGE 8200 polyolefin elastomer (obtained from the Dow

Chemical Company, Midland, Mich.) according to the methods described in United States Patent Application 2014/0234606 (Ausen), herein incorporated by reference in its entirety. The resulting net material had strand thickness (T) ranges of about 0.38-0.83 mm, opening width (W1) ranges of about 0.15-0.57 mm, opening length ranges (L1) of about 1.5-2.1 mm, and strand height (H1) ranges of about 0.60-1.24 mm.

### EXAMPLE 2

Full thickness wounds (5 cm in diameter, 1 cm deep) were surgically created on the backs of anesthetized Yucatan mini pigs (female, less than 36 weeks old and less than 35 Kg). Each wound was allowed to reach hemostasis and then wiped with gauze. A pad (5 cm diameter, 10 mm thick) of GRANUFOAM polyurethane foam (V.A.C Granufoam Dressing Medium, KCI Incorporated, San Antonio, Tex.) was placed in the wound. A conduit (3 mm I.D. plastic tubing) was connected at one end to the foam section and the other end was connected to a vacuum pump. The foam pad was covered with an occlusive, adhesive film that was adhered to skin adjacent to the wound. The vacuum pump was engaged with a pressure setting of −125 mm Hg and the foam pad was maintained in place for 3 days. Vacuum was disconnected and the foam pad was removed from the wound. The resulting exposed wound was treated by first placing the net of Example 1 (5 cm diameter section) in the wound. The net was then covered with a fresh pad (5 cm diameter, 10 mm thick) of GRANUFOAM polyurethane foam to form the test dressing. The foam pad section was connected to the vacuum pump and the test dressing was covered with an occlusive, adhesive film that was adhered to skin adjacent to the wound. The vacuum pump was engaged with a pressure setting of −125 mm Hg. The finished test dressings were maintained on the wounds for treatment times of either 3 days or 6 days.

At the end of the designated treatment period, the adhesive film and vacuum conduit were carefully removed and the peel force required to remove the test dressing from the wound was measured. Peel force was determined using a Shimpo digital force gauge (#FGV-100XY from Shimpo Instruments, Glendale Heights, Ill.) equipped with TORIEMON data acquisition software. The force gauge was mounted onto a motorized stage and positioned such that the initial peel angle was approximately 180 degrees with the retraction speed set at approximately 100 mm/minute. A blunted hook attached to the force gauge was looped through both the foam pad and netting sections to connect the test dressing to the instrument. The peel force was recorded in Newtons (N) as a function of time. The mean Peak Peel Force is reported in Table 1 for the 3 day and 6 day wear of the test dressing.

### Comparative Example 2

Comparative Example 2 differed from Example 2 in that the dressing applied on the 3^{rd} day after the initial wound creation did not include the net of Example 1. Full thickness wounds (5 cm in diameter, 1 cm deep) were surgically created on the backs of anesthetized Yucatan mini pigs (female, less than 36 weeks old and less than 35 Kg). Each wound was allowed to reach hemostasis and then wiped with gauze. A pad of GRANUFOAM polyurethane foam (5 cm diameter, 10 mm thick) was placed in the wound. A conduit (3 mm I.D. plastic tubing) was connected at one end to the foam section and the other end was connected to a vacuum pump. The foam pad was covered with an occlusive, adhesive film that was adhered to skin adjacent to the wound. The vacuum pump was engaged with a pressure setting of −125 mm Hg and the foam pad was maintained in place for 3 days. Vacuum was disconnected and the foam pad was removed from the wound. The resulting exposed wound was treated by placing a fresh pad of GRANUFOAM polyurethane foam (5 cm diameter, 10 mm thick) in the wound to form the comparative test dressing. The foam pad was connected to the vacuum pump and the comparative test dressing was covered with an occlusive, adhesive film that was adhered to skin adjacent to the wound. The vacuum pump was engaged with a pressure setting of −125 mm Hg. The finished comparative test dressings were maintained on the wounds for treatment times of either 3 days or 6 days.

At the end of the designated treatment period, the adhesive film and vacuum conduit were carefully removed and the peel force required to remove the comparative test dressing from the wound was measured. Peel force was determined using a Shimpo digital force gauge (#FGV-100XY) equipped with TORIEMON data acquisition software. The force gauge was mounted onto a motorized stage and positioned such that the initial peel angle was approximately 180 degrees with the retraction speed set at approximately 100 mm/minute. A blunted hook attached to the force gauge was looped through the foam pad to connect the comparative test dressing to the instrument. The peel force was recorded in Newtons (N) as a function of time. The mean Peak Peel Force is reported in Table 1 for the 3 day and 6 day wear of the comparative test dressing.

TABLE 1

| | Days of | Mean Peel | Number of |
|---|---|---|---|
| Example Number | Wear | Force (N) | Samples |
| Example 2 (Test Dressing) | 3 | 6.0 | 2 |
| Comparative Example 2 | 3 | 7.9 | 3 |
| (Comparative Test Dressing) | | | |
| Example 2 (Test Dressing) | 6 | 4.9 | 2 |
| Comparative Example 2 | 6 | 18.5 | 2 |
| (Comparative Test Dressing) | | | |

The peel test results demonstrate that the test dressings of Example 2 were easier to remove (required less force) than the comparative test dressings of Comparative Example 2. Following removal of the test dressings from the wounds, visual inspection showed that the test dressings of Example 2 had less in-growth of tissue into the foam layer than the comparative test dressings of Comparative Example 2.

### EXAMPLE 3

A 2.5 cm by 2.5 cm section of the net of Example 1 was prepared and the surface on one side of the net was modified by corona treatment for 15-20 seconds using a hand-held unit with rastering motion (Model BD-20 Laboratory Corona Treater, Electro-Technic Products Company, Chicago, Ill.) One surface of a 2.5 cm by 2.5 cm (12 mm thick) pad of GRANUFOAM polyurethane foam (V.A.C. Granufoam Dressing Medium, KCI Incorporated, San Antonio, Tex.) was also modified using the corona treatment procedure described above.

A 2.5 cm by 2.5 cm section of 3M #2477 Double-Coated TPE Silicone/Acrylic adhesive tape (3M Company, Maplewood, Minn.) that had been perforated (1 mm diameter perforations patterned 3 mm center-to-center) was prepared. The paper release liner was removed and the exposed adhesive surface was heated for 10-20 seconds with hot air from an electric heat gun. The adhesive tape was edge aligned and applied to the corona treated surface of the foam pad. Next, the plastic release liner was removed from the tape and the corona treated surface of the net was edge aligned and applied to the exposed adhesive surface. Hand pressure was applied to the resulting laminate for a few seconds followed by placement of a 0.46 Kg weight on the laminate overnight. The weight was removed to provide the finished laminated article.

### EXAMPLE 4

A double sided acrylic adhesive transfer tape (3M 300LSE tape #9472LE, 3M Company) was perforated through all layers in a repeating hexagonal pattern with 5 mm diameter perforations spaced 1 cm center-to-center. A 2.5 cm by 2.5 cm section of the net of Example 1 was prepared and the surface on one side of the net was modified by corona treatment for 15-20 seconds using a hand-held unit with rastering motion (Model BD-20 Laboratory Corona Treater). One surface of a 2.5 cm by 2.5 cm (12 mm thick) pad of GRANUFOAM polyurethane foam (V.A.C. Granufoam Dressing Medium) was also modified using the corona treatment procedure described above.

One of the release liners was removed from a 2.5 cm by 2.5 cm section of the double sided adhesive transfer tape and the exposed adhesive surface was heated for 10-20 seconds with hot air from an electric heat gun. The adhesive was edge aligned and applied to the corona treated surface of the foam pad. Next, the second release liner was removed and the corona treated surface of the net was edge aligned and applied to the exposed adhesive surface. Hand pressure was applied to the resulting laminate for a few seconds followed by placement of a 0.46 Kg weight on the laminate overnight. The weight was removed to provide the finished laminated article.

All references and publications cited herein are expressly incorporated herein by reference in their entirety into this disclosure. Illustrative embodiments of this invention are discussed and reference has been made to possible variations within the scope of this invention. For example, features depicted in connection with one illustrative embodiment may be used in connection with other embodiments of the invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. An article, comprising:
a network of interconnected polymeric strands; wherein each of the interconnected polymeric strands has a first surface adapted to contact a tissue site; wherein none of the interconnected polymeric strand has a feature extending from the first surface of the interconnected polymeric strands; wherein at least one of the interconnected polymeric strands is non-linear; and
a plurality of openings between adjacent interconnected polymeric strands;
wherein the article is a negative pressure wound therapy article.

2. The article of claim 1, wherein at least one of the interconnected polymeric strands is linear.

3. The article of claim 1, wherein the network comprises alternating non-linear polymeric strands and linear polymeric strands.

4. The article of claim 1, wherein the non-linear polymeric strand has a sinusoidal curve.

5. The article of claim 1, wherein the tensile strength of the article parallel to x direction is greater than the tensile strength of the article parallel to y direction.

6. The article of claim 1, wherein the tensile strength of the article parallel to x direction is more than 2.23 Mpa.

7. The article of claim 1, wherein the polymeric strands comprises an elastomeric polymer.

8. The article of claim 7, wherein the elastomeric polymer is selected from polyolefins or polyurethanes.

9. The article of claim 1, wherein aspect ratio of the openings is greater than 1:1.

10. The article of claim 1, wherein essentially all the interconnected polymeric strands are non-linear.

11. A system, comprising:
the article of claim 1; and
a reduced pressure source fluidly connected to the opening of the article to deliver the reduced pressure through the opening and to the tissue site.

12. A method, comprising:
providing the article of claim 1; and
positioning the article on a wound.

13. The method of claim 12, wherein positioning the article on the wound comprises continuously positioning the article on the wound for more than 3 days.

14. The method of claim 12, wherein positioning the article on the wound comprises continuously positioning the article on the wound for more than 4 days.

15. The method of claim 12, wherein positioning the article on the wound comprises continuously positioning the article on the wound for more than 5 days.

16. The method of claim 12, wherein positioning the article on the wound comprises continuously positioning the article on the wound for more than 6 days.

17. The method of claim 12, further comprising coupling a reduced pressure source to the article.

18. The method of claim 12, further comprising applying a reduced pressure to the wound through the article.

19. The method of claim 18, wherein applying the reduced pressure to the wound comprises activating the reduced pressure source coupled to the article.

20. The method of claim 16, wherein positioning the article on the wound comprises placing the article over the wound with the first surface facing the wound.
